(19) United States
(12) Patent Application Publication
(10) Pub. No.: US 2011/0034854 A1
(43) Pub. Date: Feb. 10, 2011

(54) PDT ASSISTED VISION CORRECTION AND SCAR PREVENTION

(75) Inventors: Wolfgang Neuberger, Labuan (MY); Volker Albrecht, Ot Bergholz-Rehbrucke (DE)

Correspondence Address: BOLESH J. SKUTNIK;CERAMOPTEC INDUSTRIES, INC., 515 SHAKER RD., EAST LONGMEADOW, MA, 01028, US

(73) Assignee: CERAMOPTEC INDUSTRIES, INC., East Longmeadow, MA (US)

(21) Appl. No.: 12/743,515

(22) PCT Filed: Nov. 20, 2008
(86) PCT No.: PCT/US08/12933

§ 371 Date: Sep. 23, 2010

Related U.S. Application Data
(60) Provisional application No. 61/003,862, filed on Nov. 20, 2007.

(51) Int. Cl. A61M 37/00; A61K 31/409; A61K 9/127; A61P 17/02

(52) U.S. Cl. 604/20; 514/410; 424/450

(57) ABSTRACT

A method and formulation are disclosed for improving the results of corneal refractive surgery, and generally reducing post-operative scarring and scarring arising from other traumas. In the vision examples, after completion of a procedure, including Photorefractive Keratotomy (PRK) or Laser-In-Situ Keratomileusis (LASIK), a photosensitizer or photosensitizer precursor is applied to the treatment site. After allowing sufficient time for the photosensitizers to accumulate among proliferating cells that occur as a result of ablation, radiation appropriate to activate the photosensitizers is administered to the treatment site. The photosensitizer is thus activated to destroy only those proliferating cells. In this way, proliferating tissue is eliminated and the cornea maintains the shape created during the surgery. As a result, instances of regression and the need for follow-up treatments, is minimized. This method is also useful for preventing post-surgical scarring that can lead to vision problems such as corneal haze. Likewise the method and formulations, presented here, are suitable for reducing post-operative scarring, and trauma cased scarring in general. For such scars, a photosensitizer or photosensitizer precursor is topically applied to the treatment site. After allowing sufficient time for the photosensitizers to attach to proliferating cells, radiation appropriate to activate the photosensitizers is administered to the treatment site. The photosensitizer is activated to destroy only those proliferating cells.

### DOMESTIC PRIORITY UNDER 35 USC 119(e).

This application claims the benefit of U.S. Provisional Application Ser. No. 61/003,862 filed Nov. 20, 2007, entitled “PDT Assisted Vision Correction and Scar Prevention” by Wolfgang Neuberger and Volker Albrecht, which is incorporated by reference herein.

### BACKGROUND OF THE INVENTION

1. Field of the Invention

The present invention relates to the field of scar repair/diminishment including that from laser vision correction. Particularly, it relates to the prevention of post-operative scarring and side-effects associated with surgical vision correction, including laser ablative corneal surgery.

2. Information Disclosure Statement

Treatment of refractive disorders has long been an active industry, particularly in the area of corrective lenses. Until the 1960's, vision correction technology had been limited to the use of corrective lenses in the form of glasses and contact lenses. Surgical vision correction techniques have been advancing rapidly, however, and such methods for vision correction have gained wide acceptance. Refractive eye surgery is the term given to those surgical procedures that act to change the light bending qualities of the cornea. Conditions like myopia (nearsightedness), hyperopia (farsightedness), and astigmatism, which result from a misshaped cornea that focuses light to a location other than an ideal location on the retina, such conditions are treated with these procedures.

The precursors to current laser ablative methods primarily consist of Radial Keratotomy (RK) and Automated Lamellar Keratoplasty (ALK). RK involves creating a number of deep radial slits in the cornea to correct myopia (nearsightedness) by making the cornea flatter and to correct astigmatism by making the cornea more rounded. This method is effective for mild cases of myopia. ALK, a treatment for myopia, involves the use of a microkeratome to make a small flap in the cornea, which is then folded away. A portion of the cornea is then removed to reshape the stroma, and the flap is replaced.

Lasers offer a significant improvement in ablative surgery. The excimer laser has become the tool of choice by practitioners because of its ability to make extremely accurate and specific alterations to the cornea with relatively little trauma. Excimer lasers emit in the UV spectrum at four major wavelengths: 193 nm (Argon Fluoride), 248 nm (Krypton Fluoride), 308 nm (Xenon Chloride), and 351 nm (Xenon Fluoride). Photorefractive Keratectomy (PRK) is a method that utilizes the excimer laser to shape the stroma by removing layers of cells. PRK is advantageous over RK because it avoids deep cuts that compromise the strength of the cornea, produces better results and reduces regression. One drawback to PRK is that the epithelial layer of cells on the cornea must be removed before the stroma (middle, thickest layer of cornea) can be ablated. This results in a corneal abrasion that usually takes a few days to heal, which presents the risk of scarring or unpredictable healing.

Laser-In-Situ Keratomileusis (LASIK) is the newest corneal ablative procedure used to correct refractive errors, and overcomes some disadvantages related to PRK. As with ALK, LASIK involves creating and lifting a flap of the cornea, exposing the stroma. Typically, an excimer laser is applied to the stroma to remove layers and reshape it. The flap is then put back into place.

A drawback to the above surgical ablative methods, especially PRK, and to a lesser extent LASIK, is that the final refractive effect of the surgery is determined not only by the ablation, but also by the eye's healing response to the destruction of corneal tissue. After the procedure is complete, the cornea produces new layers of collagen and the epithelium undergoes a hyperplasic response to the tissue destruction. These new cell layers change the shape of the cornea in a phenomenon known as regression. This process can occur months or years after treatment. Although practitioners generally attempt to take this into account before initiating the treatment, the exact amount and nature of post-operative cell proliferation cannot be predicted with certainty. As a result, the shape after proliferation may be different than what was intended, leaving the patient with inferior eyesight or necessitating further corrective procedures. However, the use of further surgeries is risky in that the healing response may be even greater and cause even further regression.

In addition to regression, the formation of new stromal collagen layers during the eye's healing response can cause scarring which manifests itself as a stromal haze that can impair a patient's vision and may reduce a patient's contrast sensitivity. Also, in PRK, there is a risk of scarring as the epithelial cells that are removed during the procedure regrow.

U.S. Pat. No. 6,162,801 by Kita discusses this hyperproliferative side effect of corneal surgery. Surgeries such as those to correct corneal refraction can result in hyperplasia, or the abnormal multiplication of normal cells in tissue due to the body's healing response. In some cases, the higher metabolism of hyperplasic cells can result in opacity and corneal refractive changes. Corticosteroids are administered after surgery to prevent hyperplasia, though their side effects include steroid-induced glaucoma and cataracts.

Kita discloses an ophthalmic composition consisting of vitamin D as the active ingredient to aid in healing and avoid symptoms such as hyperplasia that occur due to disturbed metabolism. The ophthalmic composition is administered directly to the eye after corneal surgery to regulate healing of traumatized corneal tissue.

Another attempt to improve the predictability of refractive surgery is described in U.S. Pat. No. 6,080,144 by O'Donnell, Jr. This invention attempts to both enhance the smoothness and minimize ablation zone dimensions to reduce regression and the need for over-correction. It also provides predictive formulas to account for the effects of regression due to healing. However, traumatic ablation is still a component of this method, and although improvements in results may occur due to an increased resultant smoothness, the healing response is still triggered. Because the healing response varies with individual patients, any predictive formula has inherent inaccuracies.

With the objective of diminishing the type of cellular damage that triggers a cascade of biochemical events involved in wound healing, Serdarevic, in U.S. Patent Publication No. 2008/0161780 discloses a method comprising reversible removal by chemical separation of the corneal epithelial layer, leaving a smooth surface for ablation and an epithelial flap enabling rapid hemisdesmosome reformation with firm attachment of the epithelial flap to the underlying surface. Here again, damage to the stroma and other elements of corneal anatomy is not eliminated so the healing response still occurs.

Alternatives to refractive eye surgery have been proposed to avoid problems of predictability, regression and haze that arise due to the eye's post-operative healing response. In particular, annular rings have been described that are attached to the periphery of the cornea and adjusted to change the cornea's shape to correct refractive errors. These methods avoid removing parts of the cornea, thus avoiding the onset of a healing response. Problems with these methods include added complexity and difficulty in predicting the proper shape and size of an implanted ring due to an inability to predict each individual's variable response to the procedure.

There remains a need for a relatively simple method to prevent the proliferation of corneal stroma and epithelial cells after corneal ablation to eliminate a significant cause of post-operative regression. Eliminating this cellular proliferation would improve predictability and treatment results, lower the incidence of post-operative complications, and reduce the need for subsequent procedures to further correct corneal refraction. The ability to control or eliminate the production of new stromal or epithelial cells after refractive surgery or any surgery would also be useful in preventing scarring in any case where they occur. The present invention addresses these needs.

### OBJECTIVES AND BRIEF SUMMARY OF THE INVENTION

It is an objective of the present invention to limit the proliferation of cells and thereby to eliminate instances of scarring after surgical procedures.

It is another objective of the present invention to provide a method to improve the results of corneal refractive surgery.

It is yet another objective of the present invention to limit the proliferation of cells after corneal ablation.

Briefly stated, the present invention discloses a method and formulation for improving the results of corneal refractive surgery, and generally reducing post-operative scarring and scarring arising from other traumas. In the vision examples, after completion of a procedure, including Photorefractive Keratotomy (PRK) or Laser-In-Situ Keratomileusis (LASIK), a photosensitizer or photosensitizer precursor is applied to the treatment site systemically, locally or topically. After allowing sufficient time for the photosensitizers to accumulate among proliferating cells that occur as a result of ablation, radiation appropriate to activate the photosensitizers is administered to the treatment site. The photosensitizer is thus activated to destroy only those proliferating cells. In this way, proliferating tissue is eliminated and the cornea maintains the shape created during the surgery. As a result, instances of regression and the need for follow-up treatments, is minimized. This method is also useful for preventing post-surgical scarring that can lead to vision problems such as corneal haze. Likewise the method and formulations, presented here, are suitable for reducing post-operative scarring, and trauma cased scarring in general. For such scars, a photosensitizer or photosensitizer precursor is topically, or locally applied to the treatment site. After allowing sufficient time for the photosensitizers to attach to proliferating cells, radiation appropriate to activate the photosensitizers is administered to the treatment site. The photosensitizer is activated to destroy only those proliferating cells.

The above and other objects, features and advantages of the present invention will become apparent from the following description.

### DETAILED DESCRIPTION OF PREFERRED EMBODIMENTS

Refractive surgery has become a leading procedure for the correction of vision problems due to abnormalities in the shape of the cornea. This technique has proven to be effective in its ability to alter the shape of the cornea with extreme precision and with a minimum of damage to the cornea. One drawback to refractive surgery, known as regression, occurs when the cornea changes shape after surgery, resulting in a change in the cornea's refractive properties.

One such process responsible for regression is the proliferation of cells in the cornea in response to ablation of epithelial or stromal cell layers. In this healing response, cells react to corneal damage by producing new cells. Because of the extreme precision of the procedure, and because the depth of tissue removed is on the order of microns, production of even a small number of cell layers can change the shape of the cornea and thus change the cornea's refractive properties. This results in a deterioration of the patient's vision after surgery, requiring the hassle and expense of further surgical procedures to restore the cornea to its desired shape or the need for eyeglasses. Currently, refractive surgery is performed to compensate for the healing response by over-correcting the eye. However, because each individual's healing response varies, and because cell proliferation can occur for months after the procedure, it is extremely difficult to correctly predict the amount of regression for each patient.

The present invention serves to improve the long-term results of corneal refractive surgery, especially photorefractive surgery, by destroying proliferating cells so as to prevent changes in the shape of the cornea after surgery. Utilizing a technique known as photodynamic therapy (PDT), the present invention is capable of selectively destroying only those proliferating cells produced as a result of the eye's healing response. The method leaves the remaining tissue intact, thus preserving the shape of the cornea after surgery.

PDT is a well-known method and is widely used as a cancer therapy. PDT essentially involves administering a photosensitizing agent to a treatment area to destroy abnormally proliferating cells. The photosensitizing agents, comprising photosensitizers and/or photosensitizer precursors, are unique in that they are retained in rapidly growing cells in the body longer than they are retained in normal cells. As a result, after a sufficient period of time, the body evacuates most of photosensitizers from normal cells, leaving only those photosensitive molecules that have accumulated among abnormal cells. Because photosensitive agents are unreactive until exposed to radiation of a specific wavelength, the practitioner can activate the photosensitizers after they have preferentially accumulated in abnormal cells, thus leaving healthy cells mostly unaffected.

Activation of the photosensitizer with radiation having a suitable wavelength produces singlet oxygen, oxygen radicals, and superoxides/peroxides, which in turn destroy hyperproliferative cells. Irradiation of the target site by an appropriate light source, such as a sunlamp, an argon-pumped dye laser, or more recently, diode lasers, induces the cytotoxic effect. Activated photosensitizers destroy cells by forming radicals that can initiate subsequent radical reactions to induce cytotoxic damage, or by producing singlet oxygen that subsequently produces cytotoxic oxygenated products to destroy the membranes of proliferating cells directly. Since the photosensitizers used are often based on a hydrophobic porphyrin structure, the molecules localize at cell membranes, and the oxidizing effects destroy the compartmentalization of the cell or even the cell membrane, thereby killing the cell. These highly reactive oxygen species show a limited range of action and thus only locally exhibit their destructive effects.

In a preferred embodiment of the present invention, a composition containing photosensitizing agents is topically applied to the cornea after refractive surgery has been performed. This composition may be in the form of a nonreactive cream or ointment that can be applied directly to the eye. In other embodiments, the composition can also be administered as a coating on a film. This film, which in one embodiment could be similar in shape to a contact lens and be made of any preferably flexible biocompatible material, can be inserted in the eye and worn for a sufficient period of time to allow the photosensitizers to accumulate around proliferating cells. Further embodiment is to deliver the photosensitizer by local injection directly into the eye.

The photosensitizing agents that can be used include, but are not limited to, porphyrins, dihydro- and tetrahydro-tetraphenyl porphyrins, chlorins, pheophorbides, bacteriopheophorbides, and derivatives thereof. Other known photosensitizers which could be used include hematoporphyrin derivatives, purpurins, phthalocyanines, hypocrellins, and chlorophylls. Photosensitizing agents also include precursors which in vivo naturally convert to photosensitizers, such as Alanine and Aminolevulinic Acid (ALA). From here on “photosensitizer” denotes both photosensitizers and these types of precursors. One specific photosensitizer useful in the present invention is meta-tetra(hydroxyphenyl)chlorin (“mTHPC”), also known as Temoporfin and by the trade name Foscan. mTHPC is a photosensitizer shown to be effective in PDT of cancer, especially for advanced head and neck squamous cell carcinoma. One preferred radiation wavelength for activating mTHPC is at or near 554 nm. A 554 nm wavelength penetrates well into the cornea to activate the photosensitizer accumulated in the epithelial and stromal cell without damaging the deeper retinal and other sensitive layer. Preferred formulations for use in the present invention include liposomal formulations containing appropriate photosensitizers. Liposomes are submicron, hollow vesicles consisting of hydrated, synthetic phospholipids arranged in a bilayer structure. In such formulations, non-polar, poorly water-soluble photosensitizers are encased in liposomes to facilitate absorption into the corneal or other tissue. An exemplary formulation is an aqueous pegylated liposomal solution of a dihydro-tetraphenyl porphyrin, such as, mTHPC, preferably having a mTHPC concentration of about 1.5 mg/ml of solution. For topical applications outside the ocular area, special flexible liposomal formulations as described in patent applications, having Ser. Nos. 11/800,147 and 12/226,893, may be of particular value and are incorporated herein.

After a sufficient time interval has passed to allow the photosensitizing agent to accumulate among proliferating cells, which may be as short as about 10-15 minutes or as long as an hour, the site is irradiated with radiation having a wavelength suitable to activate the photosensitizer. This time interval, which is referred to as, the drug-light interval or DLI, varies for different photosensitizers and different patients, but, as is known in the art, may be determined through methods such as fluorescence detection. Such radiation may be delivered by any known method or device including but not limited to a laser, a bare optical fiber, a lamp or other non-coherent source.

This treatment is intended to reduce unwanted cell proliferation by either reducing cell proliferation directly or indirectly by affecting the signaling processes that lead to proliferative cell growth, wherever this undesirable growth occurs. In this way, the proliferating epithelial or other corneal layer produced as a result of refractive surgery is removed, thus eliminating or minimizing the possibility of regression due to regrowth of epithelial cells.

The treatment of the present invention may be performed at varying the periods after refractive surgery. The treatment may be applied immediately, soon after, or at some other time after surgery, depending on the type of surgery. In the case of post-operative treatment of the eye after PRK, for example, it may be advantageous to delay administration of the photosensitizer until the epithelium is healed, which may be 2-3 days but will vary. PDT treatment after this period, will thus only target stromal cells that are proliferating, without interrupting or compromising healing of the epithelium. In other instances, it may be preferable to target the epithelial cells immediately or after a specific time to control epithelial healing and prevent over-proliferation of epithelial cells. For LASIK treatments, in which the epithelium is not ablated, the photosensitizer may be applied before, immediately after or soon after surgery. On the other hand, it may be desirable to delay post-operative treatment for a certain time to allow some healing after replacement of the corneal flap (as is required in procedures such as LASIK).

A similar method can be used to prevent the formation of scars during any healing process, whether in the eye or elsewhere in a patient. The procedures described herein can in particular can apply to most surgeries helping prevent post-operative scarring and its complications. Time delays after trauma causing/initiating undue proliferation of cell growth leads to ability to treat scars as they are forming or for some time thereafter.

In the case of corneal procedures, to prevent scarring that can lead to corneal haze or other complications; the same three-step method is used. Photosensitizers are applied to the surface of the eye through any known method. Sufficient time is allowed to pass so that the photosensitizers accumulate around rapidly proliferating cells. The area is then irradiated with a suitable wavelength to activate the photosensitizer and destroy the proliferating cells. The photosensitizer can be applied topically, locally or systemically before or at a time after the surgical or cosmetic procedure has been performed. Irradiation takes place after the surgical or cosmetic procedure has been performed or other trauma has occurred and after the photosensitizer has preferentially accumulated in proliferating tissue. In case of surgery utilizing conventional tools, such as in radial keratotomy, the photosensitizer may be administered well before the surgical intervention takes place to insure proper uptake. For surgical procedures utilizing lasers, such as photorefractive keratectomy and LASIK, the photosensitizer may be administered prior to surgery provided that the activation wavelength or wavelength range of the photosensitizer is significantly different from the wavelength of the surgical laser.

Having described preferred embodiments of the invention, it is to be understood that the invention is not limited to the precise embodiments, and that those skilled in the art can effect changes and modifications without departing from the scope the invention as defined in the appended claims.

What is claimed is:

1. A method for preventing cell proliferation or scar formation after surgery or other trauma, comprising the steps of:
a. applying a photosensitizing agent to a treatment area that is an object of surgery;
b. allowing sufficient time for said photosensitizer to preferentially target/associate with proliferating cells; and
c. applying suitable radiation to said treatment area after said surgery, thus destroying said proliferative cells.

2. The method for preventing cell proliferation according to claim 1, wherein it is corneal surgery; the photosensitizing agent is applied to an ocular treatment area that is an object of corneal ablative surgery; and the suitable radiation is applied to said treatment area after said corneal surgery, thus destroying said proliferative cells.

3. The method for preventing cell proliferation or scar formation after surgery according to claim 1, wherein said photosensitizing agent is chosen from the group consisting of porphyrins, dihydro-tetraphenyl porphyrins, tetrahydro-tetraphenyl porphyrins, pheophorbides, bacteriopheophorbides, alanine and aminolevulinic acid (ALA).

4. The method for preventing cell proliferation or scar formation after surgery according to claim 1, wherein said photosensitizing agent is applied in a liposomal formulation.

5. The method for preventing cell proliferation or scar formation after surgery according to claim 1, wherein said photosensitizing agent is meta-tetra (hydroxyphenyl)chlorin (“mTHPC”)

6. The method for preventing cell proliferation or scar formation after surgery, according to claim 1, wherein, said photosensitizing agent is applied topically, locally or systemically.

7. The method for preventing cell proliferation or scar formation after surgery according to claim 6, wherein a topical photosensitizer application step is accomplished by selecting from a group consisting of a cream, an ointment, an aqueous solution and a film coated with said photosensitizers.

8. The method for preventing scar formation during post-operative healing after surgery according to claim 1, wherein said surgery is corneal surgery, and wherein said surgical treatment area is a portion of the cornea that has been surgically treated.

9. The method for preventing cell proliferation or scar formation during post-operative healing after surgery according to claim 1, wherein said photosensitizer is applied to said surgical treatment area at a time before said surgery.

10. The method for preventing cell proliferation or scar formation during post-operative healing after surgery according to claim 1, wherein said photosensitizer is applied to said surgical treatment area at a preselected time after said surgery.

11. A formulation for preventing cell proliferation or scar formation after surgery or other trauma comprising a photosensitizing agent chosen from the group consisting of porphyrins, dihydro-tetraphenyl porphyrins, tetrahydro-tetraphenyl porphyrins, pheophorbides, bacteriopheophorbides, alanine and aminolevulinic acid (ALA) and an excipient or inert material to provide for systemic, local or topical application.

12. The formulation according to claim 11, wherein said photosensitizing agent is in a liposomal formulation or a pegylated liposomal formulation.
